(19) United States
(12) Patent Application Publication
(10) Pub. No.: US 2003/0165918 A1
(43) Pub. Date: Sep. 4, 2003

(54) Method for detecting nucleic acid

(75) Inventors: Kouki Nakamura, Kanagawa (JP); Hiroko Inomata, Saitama (JP); Masayoshi Kojima, Saitama (JP); Yoshihiko Makino, Saitama (JP)

Correspondence Address: SUGHRUE MION, PLLC, 2100 PENNSYLVANIA AVENUE, N.W., WASHINGTON, DC, 20037, US

(73) Assignee: FUJI PHOTO FILM CO., LTD.

(21) Appl. No.: 10/219,671

(22) Filed: Aug. 16, 2002

(30) Foreign Application Priority Data
Aug. 17, 2001 (JP) 247535/2001
Aug. 17, 2001 (JP) 247536/2001
Aug. 17, 2001 (JP) 247538/2001

(51) Int. Cl. C12Q 1/68

(52) U.S. Cl. 435/6

(57) ABSTRACT

A method for detecting a hybrid multi-stranded nucleic acid sample nucleic acid, which comprises the step of allowing the sample nucleic acid to interact with a probe nucleic acid to form a hybrid multi-stranded nucleic acid by hybridization of the probe nucleic acid and the sample nucleic acid, which further comprises the steps of allowing two or more kinds of compounds with affinity to multi-stranded nucleic acids, each having a different luminescent characteristic, to interact with the hybrid multi-stranded nucleic acid, and then detecting luminescence generated as a result of an energy transfer between the two or more kinds of the compounds with affinity to multi-stranded nucleic acids.

### FIELD OF THE INVENTION

The present invention relates to a method for convenient and accurate detection of a multi-stranded nucleic acid.

### RELATED ART

Analysis of human genome sequence has been almost completed, and it is expected that elucidation of functions of genes will rapidly advance on the basis of the information. A DNA chip is a microarray in which a large number of DNA molecules are aligned on a solid phase carrier such as slide glass, and is extremely useful for simultaneous analyses of expression, mutation, polymorphism and the like of genes. DNA-chip technologies using the DNA chip can also be applied to biomolecules other than DNA, and is expected to provide novel means for researches on developments of new drugs, developments of diagnostic and prophylactic methods for diseases, research and development of countermeasures against energy and environmental problems and the like.

Materialization of the DNA chip technologies, was first started from the development of a method for determining DNA nucleotide sequences by hybridization with oligonucleotides (SBH, sequencing by hybridization, Drmanac R. et al., Genomics, 4, p.114 (1989)). SBH successfully overcome limitation of the nucleotide sequencing methods using gel electrophoresis, however, this method was not used practically. After then, a technology for DNA chip preparation was developed, and so-called HTS (high-throughput screening) become available which enabled quick and efficient investigation of expression, mutation, polymorphism and the like of genes (Fodor S. P. A., Science, 251, p.767 (1991) and Schena M., Science, 270, p.467 (1995).

However, for practical application of the technology for DNA chip preparation, a DNA chip-preparation technique is required for alignment of a large number of DNA fragments or oligonucleotides on a surface of a solid carrier is required, and in addition, a technique is also needed for detecting hybridization of DNA fragments on a prepared DNA chip and sample nucleic acid fragments with high sensitivity and accuracy. In general, detection processes are performed by hybridization after sample nucleic acid fragments are labeled. However, the detection is complicated, because the labeling process is required for each individual samples, and additionally to the labeling reaction, a purification step is also required to separate labeled nucleic acids from unreacted remaining labeling compounds.

In order to avoid the complicated labeling step, a method has been proposed for solely detecting a multi-stranded nucleic acid formed under hybridization by using an intercalator, for example, as described in Japanese Patent Laid-open Publication (Kokai) No. 5-199898. The labeling by means of a covalent bond may denature a nucleic acid to be detected and may likely result in inhibition of precise hybridization, whilst a method using the intercalator can avoid the labeling step, and moreover, is believed to be possibly overcome the aforementioned problem.

The intercalator described in Japanese Patent Laid-open Publication No. 5-199898 is to perform detection electrochemically. The intercalator has some advantages such as possible miniaturization of an apparatus ascribed to the electrochemical detection; however, several unhandled difficulties, such as design of an electrode and immobilization of a nucleic acid on an electrode surface, may arise because a probe nucleic acid is required to be immobilized on the electrode surface for the electrochemical detection.

For these reasons, development of an intercalator has been desired which is suitable for methods for the detection using a fluorescence scanner which are widely used at present. However, intercalators known to date have a problem in that they interact with a single-stranded nucleic acid used as a sample, as well as with a multi-stranded nucleic acid, which results in insufficient selectivity. In addition to the aforementioned problem, requirements to be satisfied by intercalators for detecting a multi-stranded nucleic acid include, to achieve high sensitivity and a high S/N ratio, high efficiency in intercalation to a multi-stranded nucleic acid, large difference in signal intensity between signals generated from intercalators in intercalating and non-intercalating states. Further, intercalators in a form of a conjugate with a fluorochrome are required to have high emission efficiency of the fluorochrome, as well as suitable properties for an excitation light source and a detection wavelength of a fluorescence scanner such as an absorption maximum in a visible region. Therefore, a dye fundamental structure is desired which allows high controllability in wavelengths. Intercalators satisfying the aforementioned requirements are strongly desired. However, no intercalator has been found which is satisfactory for practical application.

### SUMMARY OF THE INVENTION

An object of the present invention is to provide a method for detecting a multi-stranded nucleic acid. More specifically, the object is to provide a method for detecting a multi-stranded nucleic acid which can be used for genetic analysis utilizing a DNA chip, DNA array or the like. The object of the present invention is to provide a method for detecting a multi-stranded nucleic with improved detection signal intensity a-d high S/N ratio.

The inventors of the present invention conducted various studies on a method in which a compound having a luminescent chromophore and selective affinity for a multi-stranded nucleic acid than for a single-stranded nucleic acid (hereinafter, a compound having this property is referred to as “a compound with affinity to multi-stranded nucleic acid”) is interacted with a hybrid multi-stranded nucleic acid produced by hybridization of a probe nucleic acid and a sample nucleic acid, and a signal generated from the compound with affinity to multi-stranded nucleic acid is detected with a high S/N ratio. As a result, they found that a multi-stranded nucleic acid was detectable with extremely high sensitivity by allowing two or more kinds of the compounds with affinity to multi-stranded nucleic acids, each of which emits light at a different wavelength, to interact with the multi-stranded nucleic acid, and then detecting luminescence generated as a result of an energy transfer between the compounds. Further, they also found that a multi-stranded nucleic acid was detectable with high sensitivity by allowing a luminescent compound with affinity to multi-stranded nucleic acid, having two or more kinds of particular chromophores, to interact with the multi-stranded nucleic acid.

Furthermore, they also found that a much higher S/N ratio was obtainable, as compared with a signal obtained from a single kind of a compound with affinity to multi-stranded nucleic acid, by allowing a combination of two or more kinds of compounds with affinity to multi-stranded nucleic acids to interact with a multi-stranded nucleic acid produced by hybridization, and then detecting a signal generated by interaction between the compounds with affinity to multi-stranded nucleic acids. They also found that, among the aforementioned combination, a higher S/N ratio was obtainable by using the compounds with affinity to multi-stranded nucleic acid having a high distinguishing ratio for a multi-stranded nucleic acid. The present invention was achieved on the basis of these findings.

The present invention thus provides a method for detecting a hybrid multi-stranded nucleic acid, which comprises the step of allowing a sample nucleic acid to interact with a probe nucleic acid to form a hybrid multi-stranded nucleic acid by hybridization of the probe nucleic acid and the sample nucleic acid, and further comprises the steps of allowing two or more kinds of compounds with affinity to multi-stranded nucleic acids, each of said compounds has a different luminescent characteristic, to interact with the hybrid multi-stranded nucleic acid, and detecting luminescence generated as a result of energy transfer between the two or more kinds of the compounds with affinity to multi-stranded nucleic acids.

According to preferred embodiments of the aforementioned method of the present invention, provided are the aforementioned method, wherein the probe nucleic acid is immobilized on a solid phase carrier; and the aforementioned method, wherein the luminescence is detected without washing after the hybrid multi-stranded nucleic acid and the compound with affinity to multi-stranded nucleic acids are allowed to interact to each other.

According to a more preferred embodiment of the aforementioned method of the present invention, provided is the aforementioned method wherein the energy transfer is a fluorescence resonance energy transfer. As preferred embodiments of the aforementioned method of the present invention, provided are the aforementioned method, wherein difference between wavelengths at maximum values in excitation spectrum and emission spectrum of the luminescence generated as a result of the energy transfer is larger than a difference between wavelengths at maximum values in excitation spectrum and emission spectrum of each of the compounds with affinity to multi-stranded nucleic acid; the aforementioned method, wherein luminescence in which a difference between wavelengths at maximum values in excitation spectrum and emission spectrum is 80 nm or more is detected; the aforementioned method, wherein luminescence in which a difference between wavelengths at maximum values in excitation spectrum and emission spectrum is 100 nm or more is detected; and the aforementioned method, wherein two or more kinds of the compounds with affinity to multi-stranded nucleic acids are used wherein each of which has a chromophore with a molecular extinction coefficient of 70,000 or more.

According to a more preferred embodiment, provided is the aforementioned method, wherein at least one of the compounds with affinity to multi-stranded nucleic acids is represented by the following general formula (I):

(IC)-[(L)ₘ-(SIG)_{q}]ₙ

(in the formula, IC represents a group having affinity for a multi-stranded nucleic acid; L represents a divalent linking group; SIG represents a chromophore that emits a detectable signal; “n” represents an integer of 2, 3 or 4; m represents 0 or 1; and “q” represents 0 or 1, provided that not all of “q” is 0 in the “n” pieces of (L)ₘ-(SIG)_{q}, and L, “m”, SIG, and “q” may be the same or different in the “n” pieces of (L)ₘ-(SIG)_{q}). Further, from another aspect, the present invention provides a compound represented by the aforementioned general formula (I) used for any of the methods mentioned above.

The present invention further provides a method for detecting a hybrid multi-stranded nucleic acid, which comprises the steps of allowing a sample nucleic acid to interact with a probe nucleic acid to form a hybrid multi-stranded nucleic acid by hybridization of the probe nucleic acid and sample nucleic acid, and allowing a compound with affinity to multi-stranded nucleic acid to interact with the hybrid multi-stranded nucleic acid and detecting luminescence from the compound to detect the hybrid multi-stranded nucleic acid, wherein the compound with affinity to multi-stranded nucleic acid is a luminescent compound having two or more kinds of chromophores, each of said chromophores has a different luminescent characteristic, and wherein at least two or more kinds of said chromophores have a difference of 80 nm or more between a maximum absorption wavelength of a chromophore of a shorter wavelength end and a maximum emission wavelength of a chromophore of a longer wavelength end.

According to a preferred embodiment of the aforementioned method of the present invention, provided are the aforementioned method, wherein the probe nucleic acid is immobilized on a solid phase carrier; the aforementioned method, wherein each of at least two or more kinds of the chromophores has a molecular extinction coefficient of 70,000 or more; and the aforementioned method, wherein the compound with affinity to multi-stranded nucleic acid is an intercalator.

The present invention further provides a method for detecting a hybrid multi-stranded nucleic acid, which comprises the steps of allowing a sample nucleic acid to interact with a probe nucleic acid to form a hybrid multi-stranded nucleic acid by hybridization of the probe nucleic acid and the sample nucleic acid, and allowing two or more kinds of compounds with affinity to multi-stranded nucleic acids, each of said compounds has a distinguishing ratio of higher than 1 for a multi-stranded nucleic acid relative to a single-stranded nucleic acid, to interact with the hybrid multi-stranded nucleic acid, and detecting interaction between said compounds to detect the hybrid multi-stranded nucleic acid, wherein at least one kind of said compound has the distinguishing ratio of 5 or more, or each of at least two kinds of said compounds has the distinguishing ratio of 3 or more.

According to a preferred embodiment of the present invention, provided are the aforementioned method, wherein the probe nucleic acid is immobilized on a solid phase carrier; the aforementioned method, wherein at least one kind of said compounds with affinity to multi-stranded nucleic acids is a luminescent compound; and the aforementioned method, wherein at least one kind of said compounds with affinity to multi-stranded nucleic acids is an intercalator.

According to the methods of the present invention, by detecting luminescence generated as a result of an energy transfer between the compounds with affinity to multi-stranded nucleic acids, or by detecting luminescence generated as a result of an energy transfer between two kinds of chromophores each having a different luminescent characteristic among two or more kinds of chromophores contained in the compound with affinity to multi-stranded nucleic acid, or further by detecting interaction between compounds with affinity to multi-stranded nucleic acids, the difference between wavelengths at maximum values in excitation spectrum and emission spectrum can be expanded, and thus detection can be achieved with an excellent S/N ratio. As a result, for detection of a sample nucleic acid using a luminescent DNA chip or the like, a hybrid multi-stranded nucleic acid such as DNA/DNA and RNA/DNA can be detected with high sensitivity without performing a complicated labeling or washing operation.

### DETAILED EXPLANATION OF PREFERRED EMBODIMENTS

In the present specification, the term “multi-stranded nucleic acid” means nucleic acid molecules associated by interaction attributable to complementary sequences (production process of the multi-stranded nucleic acid by the interaction may sometimes be referred to as “hybridization”). The multi-stranded nucleic acid is known to be in double-stranded, triple-stranded, quadruple-stranded state or the like, and the multi-stranded nucleic acid referred to in the specification include these multi-stranded states. As nucleic acids, in addition to DNA and RNA, many chemically modified compounds thereof are known, and a nucleic acid analogue called PNA, which has a polypeptide chain as a backbone, is also known. Any of these compounds fall within the multi-stranded nucleic acid referred to in the specification. Nucleic acids preferably used in the present invention are DNA, RNA, and chemically modified compounds thereof, and double-stranded nucleic acids are preferred among double-stranded, triple-stranded, quadruple-stranded nucleic acids. A multi-stranded nucleic acid produced by hybridization of a probe nucleic acid and a sample nucleic acid is referred to as “a hybrid multi-stranded nucleic acid” in the specification.

In general, energy transfer between two chromophores easily occurs between chromophores located very closely to each other, whereas energy transfer is hardly observed as a distance between chromophores increases. When two or more kinds of compounds with affinity to multi-stranded nucleic acid, each having a different luminescent characteristic, are allowed to interact with a nucleic acid, almost no energy transfer is generated between the compounds because a distance between the dyes is large, whilst in a multi-stranded nucleic acid, a probability of dense existence of chromophores become increased to allow energy transfer between the compounds.

According to the first embodiment of the method of the present invention, the method is characterized to detect a multi-stranded nucleic acid by allowing two or more kinds of compounds with affinity to multi-stranded nucleic acids, each having a different luminescent characteristic, to interact with a hybrid multi-stranded nucleic acid as mentioned above, and detecting luminescence generated as a result of an energy transfer between the two or more kinds of the compounds with affinity to multi-stranded nucleic acids. According to the second embodiment of the method of the present invention, the method is characterized to detect a multi-stranded nucleic acid by using a compound with affinity to multi-stranded nucleic acid, having two or more kinds of chromophores each having a different luminescent characteristic, and detecting luminescence generated as a result of an energy transfer between at least two kinds of the chromophores among said chromophores.

In general, where a signal is read by using a fluorescence scanner, if a difference between wavelengths at the maximum values for excitation light and a signal (emission light) generated from a compound with affinity to multi-stranded nucleic acid is small, a problem arises that the excitation light mixes in the signal. If an optical filter to cut the excitation light is adjusted in wavelength or density so as to solve the above problem, the signal is cut by the filter, and as a result, the signal obtained is dilemmatically attenuated. In order to avoid this problem, various conditions such as intensity and wavelength distribution (line width of a spectrum) of the excitation light and sharpness of the optical filter are generally required to be optimized, which is extremely complicated and makes it difficult to achieve high detection sensitivity.

According to a preferred embodiment of the first method of the present invention, luminescence can be detected under a condition that a difference between wavelengths at maximum values in excitation spectrum and emission spectrum is 80 nm or more by utilizing an energy transfer between two or more kinds of the compounds with affinity to multi-stranded nucleic acids. According to a more preferred embodiment, luminescence can be detected under a condition of the wavelength difference of 100 nm or more. Under such conditions, a signal with little noise can be detected, and extremely high detection sensitivity and detection accuracy can be achieved.

Further, luminescence generated as a result of an energy transfer between the compounds with affinity to multi-stranded nucleic acids, on the basis of interaction of the two or more kinds of the compounds with affinity to multi-stranded nucleic acids with a multi-stranded nucleic acid, can be readily detected in a state of a solution. When a multi-stranded nucleic acid obtained by hybridization of a probe nucleic acid immobilized on a solid phase carrier and a sample nucleic acid is detected, it is not necessary to remove an excess of the compound with affinity to multi-stranded nucleic acids by washing. Therefore, advantages resides in no reduction of signal intensity by washing, and moreover, avoidance of complicated washing step, per se.

A difference between wavelengths at maximum values in excitation spectrum and emission spectrum generally depends on a chromophore contained in the compound. Many chromophores, which have a large difference between wavelengths at maximum values in excitation spectrum and emission spectrum, are described in, for example, “Handbook of Fluorescent Probes and Research Chemicals, 8th Edition”, Molecular Probes, Inc. (CD-ROM published by Molecular Probes, Inc., 2001). However, with only a few exceptions, they have a molecular extinction coefficient of about several tens of thousands, and accordingly, no high detection sensitivity is expected by solely using each of the compounds, per se.

According to the first method of the present invention, two or more kinds of the compounds with affinity to multi-stranded nucleic acids are used, wherein each of which contains a chromophore emitting light at a different wavelength, and luminescence generated as a result of an energy transfer between the compounds is detected. Since a difference between wavelengths at maximum values in excitation spectrum and emission spectrum is sufficiently large in luminescence generated as a result of the energy transfer, it is not necessary to chose, as each of the compounds, a compound having a large difference between wavelengths at maximum values in excitation spectrum and emission spectrum.

In the first method of the present invention, an efficient energy transfer can be induced by preferably using two or more kinds of dyes having a large molecular extinction coefficient to improve detection sensitivity. For example, the difference between wavelengths at maximum values in excitation spectrum and emission spectrum can be made larger by using two or more kinds of the compounds with affinity to multi-stranded nucleic acids wherein each of which contains a chromophore having a molecular extinction coefficient of 70,000 or more, preferably 100,000 or more, further preferably 120,000 or more, to induce an energy transfer, preferably a fluorescence resonance energy transfer between these compounds, and thereby a degree of freedom of a detection system is also increased. An upper limit of the molecular extinction coefficient of chromophore is not particularly limited. Generally, the limit us 500,000 or lower.

Each of the two or more kinds of the compounds with affinity to multi-stranded nucleic acids used in the first method of the present invention has a distinguishable luminescent characteristic, and has a different wavelength that gives the maximum value in emission spectrum. The difference in the wavelength at the maximum value between the two or more kinds of compounds is not particularly limited so far that an energy transfer between the compounds is induced. Generally, it is desirable to chose the two or more kinds of the compounds with affinity to multi-stranded nucleic acids so that luminescence as a result of the energy transfer can be detected under a condition that a difference between wavelengths at maximum values in excitation spectrum and emission spectrum is 80 am or more, preferably 90 am or more, further preferably 100 nm or more.

Generally, the compounds with affinity to multi-stranded nucleic acids can be chosen so that a difference between a wavelength at the maximum value in excitation spectrum of a compound with affinity to multi-stranded nucleic acid of a shorter wavelength end (maximum fluorescence wavelength) and a wavelength at the maximum value in emission spectrum of a compound with affinity to multi-stranded nucleic acid of a longer wavelength end (maximum emission wavelength) becomes 80 nm or more, preferably 90 nm or more, most preferably 100 nm or more. The compound with affinity to multi-stranded nucleic acid of a shorter wavelength end means a compound of which maximum fluorescence wavelength is the lowest when the maximum fluorescence wavelengths of two or more kinds of the compounds with affinity to multi-stranded nucleic acids are compared. An upper limit is not particularly limited for a difference between the maximum fluorescence wavelength of a compound with affinity to multi-stranded nucleic acid of a shorter wavelength end and the maximum emission wavelength of a compound with affinity to multi-stranded nucleic acid of a longer wavelength end. The limit may generally be 1000 am or less.

Examples of the compound with affinity to multi-stranded nucleic acids that can be used in the first method of the present invention include, for example, nucleic acid stainers. As the nucleic acid stainers, various compounds described as nucleic acid stainers in “Handbook of Fluorescent Probes and Research Chemicals, 8th Edition” of Molecular Probes, Inc. (CD-ROM published by Molecular Probes, Inc., 2001) as well as ethidium bromide are known. Many of these dyes are known to have a low fluorescence intensity when a nucleic acid is not present, whilst have a high fluorescence intensity by an interaction with a nucleic acid.

The compounds with affinity to multi-stranded nucleic acid most preferably used in the first method of the present invention are represented by the aforementioned general formula (I). In the general formula (I), a preferred example of the IC includes a group having a planar tricyclic structure or a planar tetracyclic structure. Examples of the planar tricyclic structure include anthracene, anthraquinone, phenanthrene, phenanthroline, xanthene, carbazole, phenanthridine, phenazine, acridine and the like. Examples of the planar tetracyclic structure include structures obtained by additionally condensing a planar cyclic structure to the planar tricyclic structures exemplified above. Examples of further preferred structures include the backbones described as threading intercalators in “Analytical Chemistry (Bunseki Kagaku)”, 48, 12, pp.1095-1105 (1999).

“L” is preferably a divalent group selected from the group consisting of —C(R)(R′)—, —O—, —N(R)—, —N⁺(R)(R′)—·X—, —S(O)ᵣ—, —CO—, —S(═NR)ₜ—, and an arylene group, or a divalent group obtainable by a combination thereof. R and R′ independently represent a group selected from a hydrogen atom, an alkyl group, an aryl group, an alkoxy group, an amino group, a halogen atom, nitro group, sulfo group, carboxyl group, an ammonio group and the like. “r” represents 0, 1 or 2, and “t” represents 1 or 2.

SIG represents a chromophore that generates a detectable signal, and the group is most preferably a residue of a luminescent compound. Among the luminescent compounds, fluorescent dyes are preferred. As the fluorescent dyes, cyanine dyes, oxonol dyes, and xanthene dyes are preferred. Examples of particularly preferred fluorescent dye compounds that provide a group represented by SIG include, for example, dye compounds described in “Handbook of Fluorescent Probes and Research Chemicals 8th Edition” of Molecular Probes, Inc. (Cl)-ROM published by Molecular Probes, Inc., 2001), dye compounds described in Japanese Patent Laid-open Publication No. 2001-270957, dye portions in the compounds described in Japanese Patent Laid-open Publication No. 2001-163895, fluorescent dyes commercially available from Amersham Pharmacia such as Cy3 and Cy5 and the like.

A typical embodiment of the first method of the present invention comprises the following steps.

(1) a step of allowing a sample nucleic acid to interact with a probe nucleic acid to form a hybrid multi-stranded nucleic acid by hybridization of the probe nucleic acid and the sample nucleic acid;

(2) a step of allowing two or more kinds of the compounds with affinity to multi-stranded nucleic acids, each of which emits light at a different wavelength; to interact with the hybrid multi-stranded nucleic acid; and

(3) a step of detecting luminescence generated as a result of an energy transfer between the compounds with affinity to multi-stranded nucleic acids caused by the interaction between the two or more kinds of the compounds with affinity to multi-stranded nucleic acids and the multi-stranded nucleic acid.

In the second method of the present invention, a compound with affinity to multi-stranded nucleic acid is used which has two or more kinds of chromophores having a distinguishable luminescent characteristic. Among the chromophores, at least two kinds of the chromophores are selected to have a difference of 80 nm or more between the maximum absorption wavelength of a chromophore of a shorter wavelength end and the maximum emission wavelength of a chromophore of a longer wavelength end, thereby a fluorescence resonance energy transfer between the aforementioned at least two kinds of chromophores is induced, and luminescence resulting from the energy transfer is detected. A difference between wavelengths at maximum values in excitation spectrum and emission spectrum is sufficiently large in the luminescence generated as a result of the energy transfer. Accordingly, as each of the aforementioned at least two kinds of chromophores, it is unnecessary to chose a chromophore having a large difference between wavelengths at maximum values in excitation spectrum and emission spectrum. Each of the two or more kinds of the chromophores may preferably have a Stokes shift of 80 nm or less (a difference in a wavelength between the maximum excitation wavelength and the maximum emission wavelength).

In the second method of the present invention, chromophores having a high molecular extinction coefficient can be preferably used as the aforementioned at least two kinds of chromophores to induce efficient energy transfer, thereby detection sensitivity can be improved. For example, a compound with affinity to multi-stranded nucleic acid having two or more kinds chromophores each having a molecular extinction coefficient of, for example, 70,000 or more, preferably 100,000 or more, further preferably 120,000 or more, can be used to induce fluorescence resonance energy transfer between the chromophores, and thereby the difference between wavelengths at maximum values in excitation spectrum and emission spectrum can be expanded. In addition, a degree of freedom of the detection system is also increased. An upper limit of the molecular extinction coefficient of the chromophores is not particularly limited. Generally, the limit may be 500,000 or lower.

According to the second method of the present invention, a compound with affinity to multi-stranded nucleic acid containing two or more kinds of chromophores is used. Among the chromophores, each of at least two kinds of chromophores has distinguishable luminescent characteristic. A difference in wavelength of the maximum excitation wavelength and the maximum emission wavelength of each of the chromophores is preferably less than 80 nm, and a difference between the maximum absorption wavelength of a chromophore of a shorter wavelength end and the maximum emission wavelength of a chromophore of a longer wavelength end is 80 nm or more, preferably 90 nm or more, most preferably 100 nm or more. The chromophore of a shorter wavelength end means a chromophore of which maximum absorption wavelength is the smallest when the maximum absorption wavelengths of two or more kinds of cbromophores are compared. The luminescent characteristic, maximum absorption wavelength, maximum emission wavelength and the like of the two or more kinds of the chromophores, which are a partial structure of a compound with affinity to multi-stranded nucleic acid, can be determined by preparing luminescent compounds corresponding to the chromophores and measuring excitation spectra and emission spectra thereof. When plural of chromophores are inseparable among the two or more chromophores, such chromophores can be assumed as a single chromophore for the measurement. Therefore, those skilled in the art can readily chose two or more kinds of chromophores having a difference of 80 nm or more between the maximum absorption wavelength of a chromophore of a shorter wavelength end and the maximum emission wavelength of a chromophore of a longer wavelength end.

In the second method of the present invention, a luminescence can be detected under a condition that a difference between wavelengths at maximum values in excitation spectrum and emission spectrum is 80 nm or more, preferably 90 nm or more, most preferably 100 nm or more. Accordingly, a mixing of excitation light in a signal light can be reduced, thereby the signal can be determined with little noise. As a result, the second method of the present invention can achieve extremely high detection sensitivity and detection accuracy. An upper limit of the difference between the maximum absorption wavelength of a chromophore of a shorter wavelength end and the maximum emission wavelength of a chromophore of a longer wavelength end is not particularly limited. Generally, the limit may be 1000 nm or less.

A luminescence generated as a result of energy transfer between chromophores on the basis of the interaction of the compound with affinity to multi-stranded nucleic acid with a multi-stranded nucleic acid can be readily detected also in a state of a solution. Therefore, in the second method of the present invention, when a multi-stranded nucleic acid obtained by hybridization of a probe nucleic acid immobilized on a solid phase carrier and a sample nucleic acid is detected, it is unnecessary to remove an excess of the compound with affinity to multi-stranded nucleic acid by washing. Therefore, advantages resides in no reduction of signal intensity by washing, and moreover, avoidance of complicated washing step, per se.

The two or more kinds of the chromophores can be chosen from, for example, residues of cyanine dyes, oxonol dyes, and xanthene dyes. Examples of the chromophores most preferably used in the present invention include, for example, residues of compounds described in “Handbook of Fluorescent Probes and Research Chemicals, 8th Edition” of Molecular Probes, Inc. (CD-ROM published by Molecular Probes, Inc., 2001), residues of dyes described in Japanese Patent Laid-open Publication No. 2001-270957, residues corresponding to dye portions in the compounds described in Japanese Patent Laid-open Publication No. 2001-163895, residues of fluorescent dyes commercially available from Amersham Pharmacia such as Cy3 and Cy5 and the like.

Multi-stranded nucleic acid affinity compounds most preferably used in the second method of the present invention are represented by the following general formula (II):

(IC)-[(L)ₘ-(SIG)_{q}]ₙ

(in the formula, IC represents a group having affinity for a multi-stranded nucleic acid; L represents a divalent linking group; SIG represents a luminescent chromophore; “n” represents an integer of from 2 to 10, preferably 2, 3 or 4; “m” represents 0 or 1; and “q” represents 0 or 1, provided that, among the “n” pieces of (L)ₘ-(SIG)_{q}, q is 1 in at least two or more of the groups, and among “n” pieces of (L)ₘ-(SIG)_{q}, L, m, SIG and q may be the same or different).

Ia the general formula (II), preferred examples of the IC include groups having a planar tricyclic structure or a planar tetracyclic structure. Examples of the planar tricyclic structure include anthracene, anthraquinone, phenanthrene, phenanthroline, xanthene, carbazole, phenanthridine, phenazine, acridine and the like Examples of the planar tetracyclic structure include those obtained by additionally condensing a planar cyclic structure to the planar tricyclic structures exemplified above. Examples of more preferred structures include backbone structures described as threading intercalators in “Analytical Chemistry”, 48, 12, pp.1095-1105 (1999).

“L” is preferably a divalent group selected from the group consisting of —C(R)(R′)—, —O—, —N(R)—, —N⁺(R)(R′)—·X—, —S(O)ᵣ—, —CO—, —S(═NR)ₜ—, and an arylene group, or a divalent group obtainable by a combination thereof. R and R′ independently represent a group selected from a hydrogen atom, an alkyl group, an aryl group, an alkoxy group, an amino group, a halogen atom, nitro group, sulfo group, carboxyl group, an ammonio group and the like. “r” represents 0, 1 or 2, and “t” represents 1 or 2.

The compounds represented by the general formula (II) have two or more kids of “SIG”. In order to induce resonance an energy transfer between these SIGs, the minimum number of atoms in the SIG involved in the resonance energy transfer is preferably 100 or less, more preferably 80 or less, most preferably 60 or less. “L” is preferably designed from the aforementioned viewpoint.

SIG represents a luminescent chromophore, and is preferably a residue of a fluorescent compound. The compounds represented by the general formula (II) have two or more kinds of SIGs. In order to reduce a mixing of an excitation light in a signal light, as for at least two kinds of these SIGs, a difference may be 80 nm or more between maximum absorption wavelength of a chromophore of a shorter wavelength end and maximum emission wavelength of a chromophore of a longer wavelength end, preferably 90 nm or more, most preferably 100 nm or more.

SIG in the general formula (II) can be chosen from, for example, residues of cyanine dyes, oxonol dyes, and xanthene dyes. Examples of particularly preferred SIG include residues of compounds described in “Handbook of Fluorescent Probes and Research Chemicals, 8th Edition” of Molecular Probes, Inc. (CD-ROM published by Molecular Probes, Inc., 2001), residues of dyes described in Japanese Patent Laid-open Publication No. 2001-270957, residues corresponding to dye portions in the compounds described in Japanese Patent Laid-open Publication No. 2001-163895, fluorescent dyes commercially available from Amersham Pharmacia such as Cy3 and Cy5 and the like.

A typical embodiment of the second method of the present invention comprises the following steps:

(1) a step of allowing a sample nucleic acid to interact with a probe nucleic acid to form a hybrid multi-stranded nucleic acid by hybridization of the probe nucleic acid and the sample nucleic acid;

(2) a step of allowing a compound with affinity to multi-stranded nucleic acid having two or more kinds of chromophores (among the aforementioned chromophores, each of two kinds of chromophores has a distinguishable luminescent characteristic, and a difference between the maximum absorption wavelength of a chromophore of a shorter wavelength end and the maximum emission wavelength of a chromophore of a longer wavelength end is. 80 nm or more) to interact with the hybrid multi-stranded nucleic acid; and

(3) a step of detecting luminescence generated as a result of energy transfer between the two kinds of chromophores in the compound with affinity to multi-stranded nucleic acid on the basis of an interaction between the compound with affinity to multi-stranded nucleic acid and the multi-stranded nucleic acid.

In the third method of the present invention, the term “distinguishing ratio for a single-stranded nucleic acid and a multi-stranded nucleic acid” used for the compound with affinity to multi-stranded nucleic acid means a ratio of affinity of the compound with affinity to multi-stranded nucleic acid for a multi-stranded nucleic acid based on affinity of said compound for a single-stranded nucleic acid (namely, relative affinity of the compound for a multi-stranded nucleic acid when affinity of the compound for a single-stranded nucleic acid is taken as 1). The compound with affinity to multi-stranded nucleic acids used in the third method of the present invention are those having the aforementioned distinguishing ratio higher than 1.

When double-stranded DNA is used as the multi-stranded nucleic acid, the aforementioned distinguishing ratio can be measured according to the following method. A compound with affinity to multi-stranded nucleic acid to be measured is allowed to interact with single-stranded DNA immobilized on a solid phase carrier and optionally washed as required, and then a signal is detected. Then, separately, the same DNA as the above is immobilized on a solid phase carrier and a DNA having a sequence complementary to the DNA is hybridized, and the same compound with affinity to multi-stranded nucleic acid is allowed to interact with the hybrid to detect a signal. The distinguishing ratio can be represented as (Signal intensity detected for double-stranded DNA)/(Signal intensity detected for single-stranded DNA). For the signal detection, a widely used fluorescence scanner, electric current values used in electrochemical detection described in Japanese Patent Laid-open Publication No. 5-199898, SPR (surface plasmon resonance) described in Japanese Patent Laid-open Publication Nos. 11-332595 and 2001-183292 and the like may be used. An upper limit of the distinguishing ratio is not particularly limited, and a higher ratio is more desirable. In general, the distinguishing ratio is 108 or less, preferably about 10% or less.

As for the interaction between the compound with affinity to multi-stranded nucleic acids, it is preferable to utilize an interaction whose strength is remarkably increased when the compound with affinity to multi-stranded nucleic acids approach to each other, and detect a signal caused by the interaction. As the interaction, for example, electron transfer, energy transfer or the like can be advantageously utilized. Where the electron transfer is utilized, radical and current values are preferred as a signal to be detected. Where the energy transfer is utilized, luminescence is preferably detected. In the present invention, any of the above examples can be preferably used. Detection of luminescence caused by energy transfer is most preferred.

Examples of the compounds with affinity to multi-stranded nucleic acids used in the third method of the present invention include, for example, nucleic acid stainers. As the nucleic acid stainers, various compounds described as nucleic acid stainers in “Handbook of Fluorescent Probes and Research Chemicals, 8th Edition” of Molecular Probes, Inc. (CD-ROM published by Molecular Probes, Inc., 2001) as well as ethidium bromide are known. Many of these dyes are known to have a low fluorescence intensity when a nucleic acid is not present, whilst to exhibit a high fluorescence intensity by interaction with a nucleic acid. Further examples of the compound with affinity to multi-stranded nucleic acids most preferably used in the third method of the present invention include the compounds represented by the aforementioned general formula (I).

A typical embodiment of the third method of the present invention comprises the following steps:

(1) a step of allowing a sample nucleic acid to interact with a probe nucleic acid to form a hybrid multi-stranded nucleic acid by hybridization of the probe nucleic acid and the sample nucleic acid;

(2) a step of allowing two or more kinds of compounds with affinity to multi-stranded nucleic acids to interact with the hybrid multi-stranded nucleic acid; and

(3) a step of detecting a signal deriving from an interaction between the compound with affinity to multi-stranded nucleic acids generated when the two or more kinds of the compounds with affinity to multi-stranded nucleic acids and the multi-stranded nucleic acid become close to each other.

For carrying out the first to third methods of the present invention, the probe nucleic acid may preferably be immobilized on a solid phase carrier, although the probe nucleic acid may exist in a solution. As the solid phase carrier, a hydrophobic carrier or a carrier with low hydrophilicity is preferred. Further, a carrier with low planarity having a rough surface can be preferably used. Examples of the material of the solid phase carrier include glass, cement, ceramics such as pottery or new ceramics, polymers such as polyethylene terephthalate, cellulose acetate, polycarbonate of bisphenol A, polystyrene and polymethyl methacrylate, silicon, activated carbon, porous substances such as porous glass, porous ceramics, porous silicon, porous activated carbon, woven fabric, non-woven fabric, base paper, short fiber, and membrane filter. The shape and size of the solid phase carrier are not particularly limited, and the carrier may be in plate-like, sphere-like, rod-like shape or the like, and may have a size of from nanometer to centimeter order. The pore size of the porous substances is preferably, for example, in the range of from 2 to 1000 nm, particularly preferably in the range of from 2 to 500 nm. From viewpoints of easiness of surface treatment and readiness of analysis by an electrochemical method, the material of the solid phase carrier is most preferably glass or silicon. Where a plate-like solid phase carrier is used (hereinafter, a solid phase carrier in such a shape may be referred to as “a substrate”, and examples where the solid phase carrier is a substrate may be explained as a preferred embodiment of the present invention), the solid phase carrier preferably has a thickness in the range of from 100 to 2000 μm.

As the method for immobilizing a probe nucleic acid on a solid phase carrier, an appropriate method can be chosen depending on a type of the nucleic acid fragment and a type of the solid phase carrier (Protein, Nucleic acid and Enzyme, Vol. 43, No. 13, pp.2004-2011 (1998)). For example, where the nucleic acid fragment is cDNA or a PCR product, applicable methods include a method of electrostatically coupling the fragment by utilizing charge of DNA to a substrate subjected to a surface treatment with a cation such as polylysine, polyethylenimine, or polyalkylamine. A layer of hydrophilic polymer substance or the like having an electric charge or a layer comprising a crosslinking agent may further be provided on the surface-treated substrate. Further, depending on a type of the substrate, a hydrophilic polymer or the like can be immersed in the substrate, and a substrate subjected to such treatment can also be preferably used. By applying the surface treatment, electrostatic interaction between a hydrophobic substrate or weakly hydrophilic substrate and a nucleic acid fragment can be enhanced. For that purpose, a slide glass is preferably used as the substrate from viewpoints of easiness of the surface treatment and readiness of analysis.

When synthetic nucleotides are immobilized, applicable methods include a method of directly synthesizing nucleotides on a substrate, or a method of synthesizing an oligomer whose end is introduced beforehand with a functional group for forming a covalent bond, and then covalently bonding the oligomer to a surface-treated substrate. Examples of the functional group include an amino group, an aldehyde group, a mercapto group, biotin and the like. As the substrate, glass or silicon is preferably used, and a known silane-coupling agent is preferably used for the surface treatment of glass or silicon.

Although the nucleic acid immobilized on the substrate may be a sample nucleic acid to be detected, the following explanation will be made, only for a sake of convenience, as for examples wherein a nucleic acid immobilized on the substrate is a probe nucleic acid (hereafter “DNA fragment” will be explained as a typical example of the probe nucleic acid) and a nucleic acid allowed to interact with the probe nucleic acid is a sample nucleic acid.

DNA fragments can be classified into two groups depending on purposes. In order to investigate gene expression, polynucleotides such as cDNA, a part of cDNA and EST are preferably used. Functions of these polynucleotides may be unknown, and they are generally prepared by amplifying a DNA fragment by PCR using a cDNA library, genome library, or the whole genome as a template based on a sequence registered in a database. DNA fragments that are not amplified by PCR can also be preferably used. Further, in order to investigate gene mutation or polymorphism, it is preferable to synthesize various oligonucleotides corresponding to a mutation or a polymorphism based on known sequences as a standard and use the synthesized product. Further, for nucleotide sequence analysis, 4ⁿ (n is number of bases) kinds of synthesized oligonucleotides are preferably used. The nucleotide sequence of the DNA fragment is preferably determined beforehand by an ordinary nucleotide sequencing method. The DNA fragment is preferably a 2- to 50-mer; most preferably 10- to 25-mer.

Spotting of the DNA fragments on a solid phase carrier is preferably performed by, for example, preparing an aqueous liquid containing the DNA fragments by dissolving or dispersing the fragments in an aqueous medium, introducing this aqueous liquid into each well of a 96-well or 384-well plastic plate, and dropping the introduced aqueous liquid on a solid phase carrier surface by using a spotting device or the like.

In order to prevent the DNA fragments from drying after the spotting, a substance having a high-boiling point may be added to the aqueous liquid in which the DNA fragments are dissolved or dispersed. As the substance having a high-boiling point, a substance is preferred that is dissolvable in the aqueous liquid in which the DNA fragments are dissolved or dispersed, is free from inhibition of hybridization of the DNA fragments and the sample nucleic acid, and is not viscous. Examples of such substances include glycerin, ethylene glycol, dimethyl sulfoxide, and low molecular hydrophilic polymers. Examples of the hydrophilic polymers include, polyacrylamide, polyethylene glycol, sodium polyacrylate and the like. The molecular weight of the polymer is preferably in the range of 10³-10⁵. As the substance having a high-boiling point, glycerin or ethylene glycol is more preferably used, and glycerin is most preferably used. The concentration of the substance having a high-boiling point in the aqueous liquid of the DNA fragments is preferably in the range of 0.1 to 2% by volume, most preferably in the range of 0.5 to 1% by volume. Further, for the same purpose, it is also preferable to place the solid phase carrier after the spotting of the DNA fragments under conditions of a humidity of 90% or more and a temperature of 25 to 50° C.

After the DNA fragments are spotted on the solid phase carrier, a post-treatment by using ultraviolet rays, sodium borohydride, Schiff reagent or the like may be applied. For the post treatment, plural kinds of treatments maybe performed in combination, and a heat treatment and ultraviolet irradiation or the like are most preferably performed in combination. Incubation is also preferably carried out after the spotting. After the incubation, it is preferable to remove unimmobilized DNA fragments by washing.

The density of the DNA fragments is preferably in the range of 10² to 10⁵ kinds/cm² on the solid phase carrier surface. The amount of the DNA fragments is preferably in the range of 1 to 10⁻¹⁵ moles, and the mass is preferably a few ng or less. By the spotting, the aqueous liquid containing the DNA fragments is immobilized on the solid phase carrier surface in a shape of a dot. The shape of the dots is usually a substantially circular form. For a quantitative analysis of gene expression or analysis of a single base mutation, it is important that the dot shape remains unchanged. The distance between the dots is preferably in the range of 0 to 1.5 mm, most preferably in the range of 100 to 300 μm. The size of one dot is preferably in the range of 50 to 300 μm in diameter. The volume of the aqueous liquid to be spotted is preferably in the range of 100 pL to 1 μL, most preferably in the range of 1 to 100 nL.

The lifetime of a solid phase carrier immobilized with DNA fragments (hereinafter, referred to as “a DNA chip”), which is prepared through the aforementioned steps, is several weeks for a cDNA chip immobilized with cDNAs, and is still longer for an oligo DNA chip immobilized with oligo DNAs. These DNA chips can be used in monitoring of gene expression, nucleotide sequencing, mutation analysis, polymorphism analysis and the like.

As the sample nucleic acid, a sample containing a DNA fragment or RNA fragment of which sequence or function is unknown is preferably used. For the purpose of investigation of gene expression, the sample nucleic acid is preferably isolated from a cell or tissue sample of an eukaryote. When the sample is a genome, the sample nucleic acid can be isolated from any tissue samples except for erythrocytes. Any tissues except for erythrocytes may preferably be peripheral blood lymphocytes, skin, hair, sperm or the like. When the sample is mRNA, the sample is preferably extracted from a tissue sample in which mRNA is expressed. mRNA is preferably converted into cDNA by reverse transcription to take up dNTP (“dNTP” refers to a deoxyribonucleotide having a base of adenine (A), cytosine (C), guanine (G) or thymine (T)). As dNTP, dCTP is preferably used from a viewpoint of chemical stability. An amount of mRNA required for one hybridization varies depending on a liquid volume or a labeling method. The amount may preferably be a few μg or less. When DNA fragments on a DNA chip is oligo DNAs, the sample nucleic acid is preferably made to lower molecules beforehand. Since it is difficult to selectively extract mRNA from prokaryotic cells, it is preferable to label total RNA.

Hybridization can be performed by introducing an aqueous liquid dissolving or dispersing a sample nucleic acid into each well of a 96-well or 384-well plastic plate and spotting the liquid at positions of DNA fragments on the DNA chip prepared as described above. The volume of the aqueous liquid to be spotted is preferably, for example, in the range of 1 to 100 nL. Hybridization can be usually performed at a temperature in the range of room temperature to 70° C. for a reaction time in the range of 6 to 20 hours.

After completion of the hybridization, the chip is preferably washed with a mixed solution of a surfactant and a buffer to remove unreacted sample nucleic acid. As the surfactant, sodium dodecylsulfate (SDS) is preferably used. As the buffer, citrate buffer, phosphate buffer, borate buffer, Tris buffer, Good's buffer and the like can be used. Citrate buffer is most preferably used. When a compound represented by the general formula (I) or (II) is allowed to coexist during the hybridization and react with a hybrid multi-stranded nucleic acid, the chip may be washed in the same manner as described above. Where luminescence generated as a result of energy transfer is detected by utilizing a fluorescence resonance energy transfer phenomenon or the like, optical detection can be performed without washing.

Hybridization using a DNA chip is characterized in that a very small amount of a sample nucleic acid is used. For this reason, optimal conditions for hybridization should be applied depending on lengths of DNA fragments immobilized on a solid phase carrier and a type of the sample nucleic acid. For a gene expression analysis, it is preferable to perform hybridization for a long period of time so that low expression genes can also be satisfactorily detected. For detection of single base mutation, it is preferable to perform hybridization for a short period of time.

The step of allowing a compound with affinity to multi-stranded nucleic acid to interact with a hybrid multi-stranded nucleic acid may be performed simultaneously with the aforementioned hybridization, or said step may be performed after the hybridization. Where two or more kinds of the compounds with affinity to multi-stranded nucleic acids are allowed to interact with a hybrid multi-stranded nucleic acid, all of the compounds with affinity to multi-stranded nucleic acids may be mixed during the hybridization step, or alternatively, one or more kinds of the compound with affinity to multi-stranded nucleic acids may be mixed during the hybridization step, and after the hybridization, one or more kinds of the compounds with affinity to multi-stranded nucleic acids may be mixed which are different from the aforementioned compounds. The temperature during the interaction step is not particularly limited. When a compound with affinity to multi-stranded nucleic acid is added during hybridization, the step may be performed at the hybridization temperature explained above. When a compound with affinity to multi-stranded nucleic acid is added after hybridization, it is necessary to apply a temperature at which a hybrid multi-stranded nucleic acid is not dissociated. For example, the temperature is preferably in the range of 10 to 70° C., more preferably in the range of 25 to 65° C.

As the solvent used when a compound with affinity to multi-stranded nucleic acid is brought into contact with a hybrid multi-stranded nucleic acid, water and various buffers, as well as a mixed solvent of a water-miscible organic solvent and water can be appropriately used. Preferred examples of the water-miscible organic solvent are dimethyl sulfoxide, dimethylformamide, methanol, ethanol, ethylene glycol, glycerine and so forth. Further, buffers mixed with these organic solvents can also be preferably used.

An amount of the compound with affinity to multi-stranded nucleic acid varies depending on conditions such as type of a probe nucleic acid used and number of nucleotides. It is preferable to adjust the solution concentration so that the number of molecules of about 10⁻³-10⁷ times, more preferably about 10⁻²-10⁵ times, of the total number of bases contained in the probe nucleic acid are supplied. When two or more kinds of the compounds with affinity to multi-stranded nucleic acids are used in the methods of the present invention, each of the compounds with affinity to multi-stranded nucleic acid can be used in an amount within the range mentioned above, and the amount can be adjusted so that detection sensitivity becomes optimal depending on molecular extinction coefficient and emission quantum yield of a chromophore of each compound.

After the compound with affinity to multi-stranded nucleic acid is brought into contact with the hybrid multi-stranded nucleic acid, washing is preferably performed to remove an excess amount of the compound with affinity to multi-stranded nucleic acid. The washing can be performed by a procedure similar to the washing after the hybridization. Optical detection is achievable without performing the washing in the methods of the present invention, and omission of the washing is also preferred.

An interaction between the compound with affinity to multi-stranded nucleic acids can be detected by an optical means. Where the hybrid multi-stranded nucleic acid is detected in a solution system, a usual fluorometer can also be used, or a fluorescence scanner is also preferably used from viewpoints of ability to simultaneously detect a large number of nucleic acids and high sensitivity. Further, quantification of fluorescence may be carried out by a conventional method by using a fluorescence laser scanner for a solid phase carrier dried after hybridization or in the presence of an aqueous solvent, or the measurement may be performed by the cooled CCD (charge coupled device) method for a solid phase carrier covered with cover glass to prevent the carrier from drying.

For carrying out the method of the present invention, a nucleic acid sample may be labeled by an appropriate means beforehand, and then a hybrid multi-stranded nucleic acid may be detected by a combination of the method of the present invention with detection using a compound with affinity to multi-stranded nucleic acid. For example, the nucleic acid sample may be labeled with a fluorescent dye. Alternatively, labeling means such as an RI method or non-RI method such as a biotin method or chemiluminescence method can be employed. Any of fluorescent substances that can bind to a base moiety of a nucleic acid can be used. For example, cyanine dyes (for example, Cy3, Cy5 etc. in the CyDye™ series), Rhodamine 6G reagent, N-acetoxy-N2-acetylaminofluorene (AAF) or AAIF (iodine derivative of AAF) can be used.

### EXAMPLES

The present invention will be explained more specifically with reference to the following examples. However, the scope of the present invention is not limited to these examples.

### Example 1

### Detection of DNA Hybrid

(1) Preparation of DNA Fragment-Immobilized Slide

Slide glass (25 mm×75 mm) was immersed in a 2 weight % solution of aminopropylethoxysilane (Shin-Etsu Chemical Co., Ltd.) in ethanol for 10 minutes, taken out from the solution, washed with ethanol and dried at 110° C. for 10 minutes to prepare Silane compound-coated slide (A). Then, the Silane compound-coated slide (A) was immersed in a 3 mass % solution of Compound VS-1 for 10 minutes, taken out from the solution, washed with ethanol and dried at 120° C. for 15 minutes to prepare VS-1-coated slide (13).

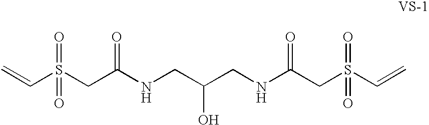

(2) Detection of Hybrid Multi-Stranded Nucleic Acid

An aqueous liquid containing DNA of SEQ ID NO: 1 mentioned in Sequence Listing, of which 3′ end was modified with an amino group, dispersed in 0.1 M carbonate buffer (pH 9.8, 1×10⁻⁶ M, 1 μL) was spotted on Slide (B) obtained in the above (1) to prepare Slide (C). Immediately after the spotting, the slide was left at 60° C. and humidity of 90% for 1 hour and then heated at 120° C. for 20 minutes. This slide was successively washed twice with a mixed solution of 0.1 mass % SDS (sodium dodecylsulfate) and 2×SSC (2×SSC: a solution obtained by diluting a stock solution of SSC 2-fold, SSC: standard saline citrate buffer) and once with a 0.2×SSC aqueous solution. Then, the slide after the aforementioned washing was immersed in a 0.1 M glycine aqueous solution (pH 10) for 1 hour and 30 minutes, washed with distilled water and dried at room temperature to obtain Slide (D) on which the DNA fragments were immobilized.

60-mer DNA having a sequence complementary to the aforementioned DNA sequence was dispersed in a hybridization solution (mixed solution of 4×SSC and 10 mass % SDS, 20 μL), added with a compound with affinity to multi-stranded nucleic acid (shown below as Comparative Example 1, Invention 1, and Invention 2, each 1 μL of 0.1 mM solution in dimethyl sulfoxide) and applied to Slide (D) obtained above. After the surface of Slide (D) was protected with cover glass for microscope, the slide was incubated in a moisture chamber at 60° C. for 10 hours. The slide glass was measured without washing by using a fluorescence scanning apparatus. Excitation was attained at an excitation wavelength as close as possible to a maximum absorption wavelength of a dye of shorter wavelength end, and the detection wavelength was controlled by adjusting a filter so as to obtain a maximum value.

Then, this slide was washed with a mixed solution of 0.1 mass % SDS and 2×SSC, centrifuged at 600 rpm for 20 seconds and dried at room temperature. Fluorescence intensity on each slide glass surface was measured by the fluorescence scanning apparatus. Excitation was attained at a wavelength as close as possible to a maximum absorption wavelength of a dye of shorter wavelength end, and the detection wavelength was controlled by adjusting a filter so as to obtain a maximum value. Further, the same experiment was performed without adding the 60-mer DNA fragment having a complementary sequence.

In Table 1, fluorescence intensities of the samples are shown as values relative to fluorescence intensity of the sample of Comparative Example 1, which was taken as 100, measured after washing with the addition of the complementary strand. As a result, Comparative Example 1 without washing gave no signal and high background, because no fluorescence resonance energy transfer occurred. Whilst in the samples of Invention 1 and Invention 2, signals were detectable without washing, and the background was low even after washing and an excellent SIN ratio was obtained. In Comparative Example 1, the compound of Comparative Example 1 was excited and luminescence from the compound was detected. For the samples of Invention 1 and Invention 2, respective compounds of a shorter wavelength end were excited and luminescence from each compound of a longer wavelength end was detected.

TABLE 1

| | | Fluorescence intensity |
|---|---|---|
| | Signal fluorescence | without complementary |
| Sample | intensity | strand |
| Comparative Example 1 | Strong (immeasurable) | Strong (immeasurable) |
| (before washing) | | |
| Comparative Example 1 | 100 | 31 |
| (after washing) | | |
| Invention 1 (before | 57 | 9 |
| washing) | | |
| Invention 1 (after washing) | 31 | 3 |
| Invention 2 (before | 70 | 16 |
| washing) | | |
| Invention 2 (after washing) | 38 | 5 |

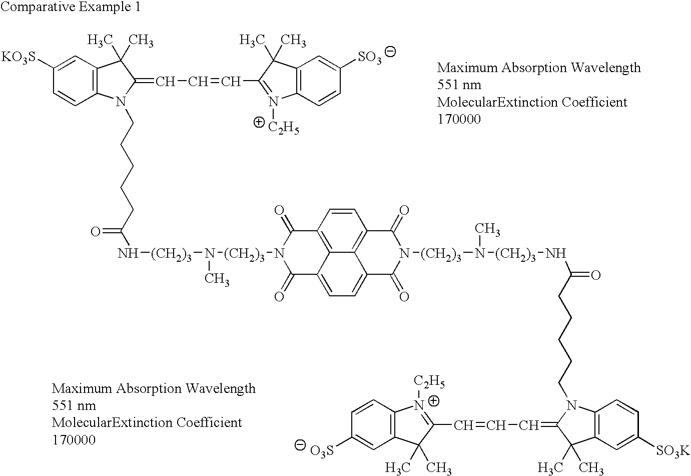

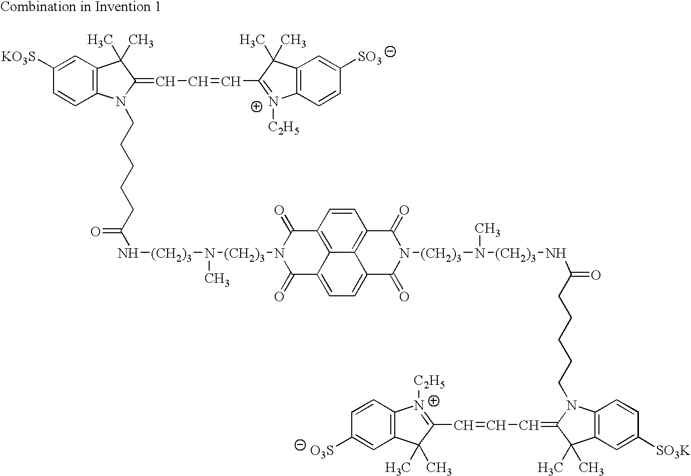

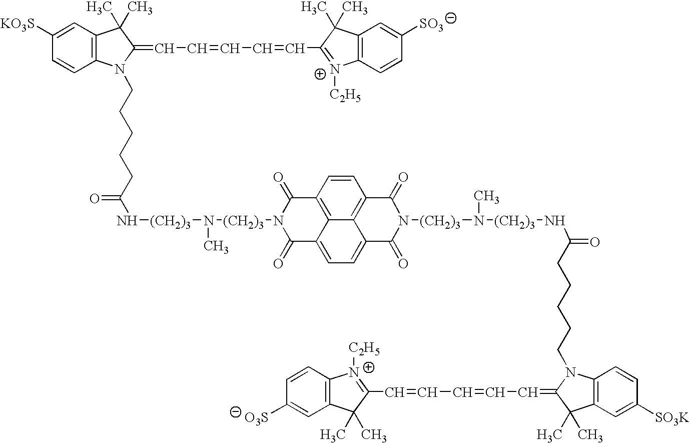

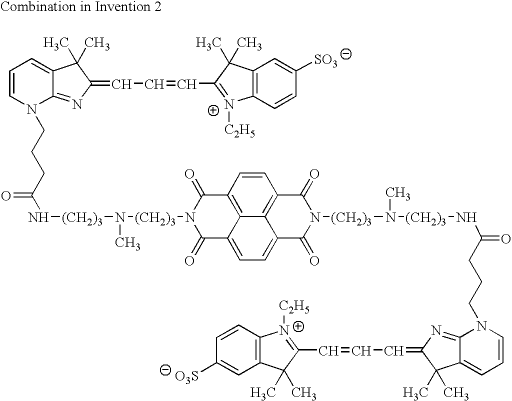

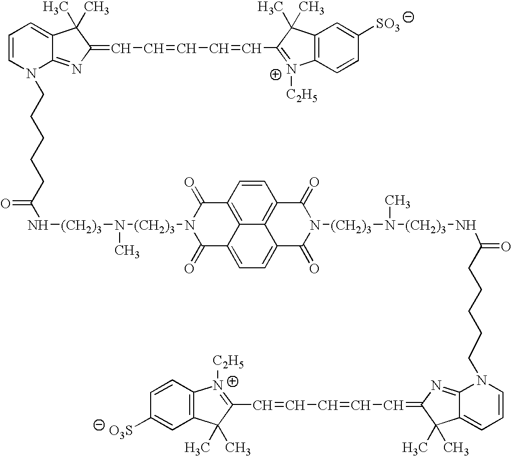

### Example 2

### Detection of DNA/RNA Hybrid

The same experiment as in Example 1 was performed except that 40-mer oligo-deoxy-A was immobilized on the slide glass surface instead of the 60-mer DNA fragment and oligo-U was used as the complementary sequence. As a result, results similar to those in Example 1 were obtained

### Example 3

### Detection of DNA Hybrid

(1) Preparation of DNA Fragment-Immobilized Slide

Silane compound-coated slide (A) and VS-i-coated slide (B) were prepared in the same manner as in Example 1.

(2) Detection of Hybrid Multi-Stranded Nucleic Acid

An aqueous liquid containing DNA of SEQ ID NO: 1 mentioned in Sequence Listing, of which 3′ end was modified with an amino group, dispersed in 0.1 M carbonate buffer (pH 9.8, 1×10⁻⁶ M, 1 μL) was spotted on Slide (B) obtained in the above (1) to prepare Slide (C). Immediately after the spotting, the slide was left at 60° C. under humidity of 90% for 1 hour and then heated at 120° C. for 20 minutes. This slide was successively washed twice with a mixed solution of 0.1 mass % SDS (sodium dodecylsulfate) and 2×SSC (2×SSC: a solution obtained by diluting a stock solution of SSC 2-fold, SSC: standard saline citrate buffer) and once with a 0.2×SSC aqueous solution. Then, the slide after the aforementioned washing was immersed in a 0.1 M glycine aqueous solution (pH 10) for 1 hour and 30 minutes, washed with distilled water and dried at room temperature to obtain Slide (D) on which the DNA fragments were immobilized.

60-mer DNA having a sequence complementary to the aforementioned DNA sequence was dispersed in a hybridization solution (mixed solution of 4×SSC and 10 mass % SDS, 20 μL), added with a compound with affinity to multi-stranded nucleic acid (shown below as Comparative Example 2, Comparative Example 3, Invention 3 and Invention 4, 1 μL each of 0.1 mM solution in dim-ethyl sulfoxide) and applied to Slide (D) obtained above. After the surface or Slide (D) was protected with cover glass for microscope, the slide was incubated in a moisture chamber at 60° C. for 10 hours. Then, this slide was washed with a mixed solution of 0.1 mass % SDS and 2×SSC, centrifuged at 600 rpm for 20 seconds and dried at room temperature. Further, the same experiment was performed without adding the 60-mer DNA fragment having a complementary sequence.

Fluorescent intensity of each slide glass was measured by a fluorescence scanning apparatus. Excitation was attained at a wavelength as close as possible to a maximum absorption wavelength of a chromophore on the short wavelength side, and the detection wavelength was controlled by adjusting a filter so as to obtain a maximum value. As a result, in Comparative Examples 2 and 8, the fluorescence intensity was generally low irrespective of presence or absence of a complementary strand in comparison with the results of Invention 3 and Invention 4. Whilst the samples of Invention 3 and Invention 4 was found to exhibit high fluorescence intensity and also gave a more significant difference in fluorescence intensity depending on the presence or absence of the complementary strand.

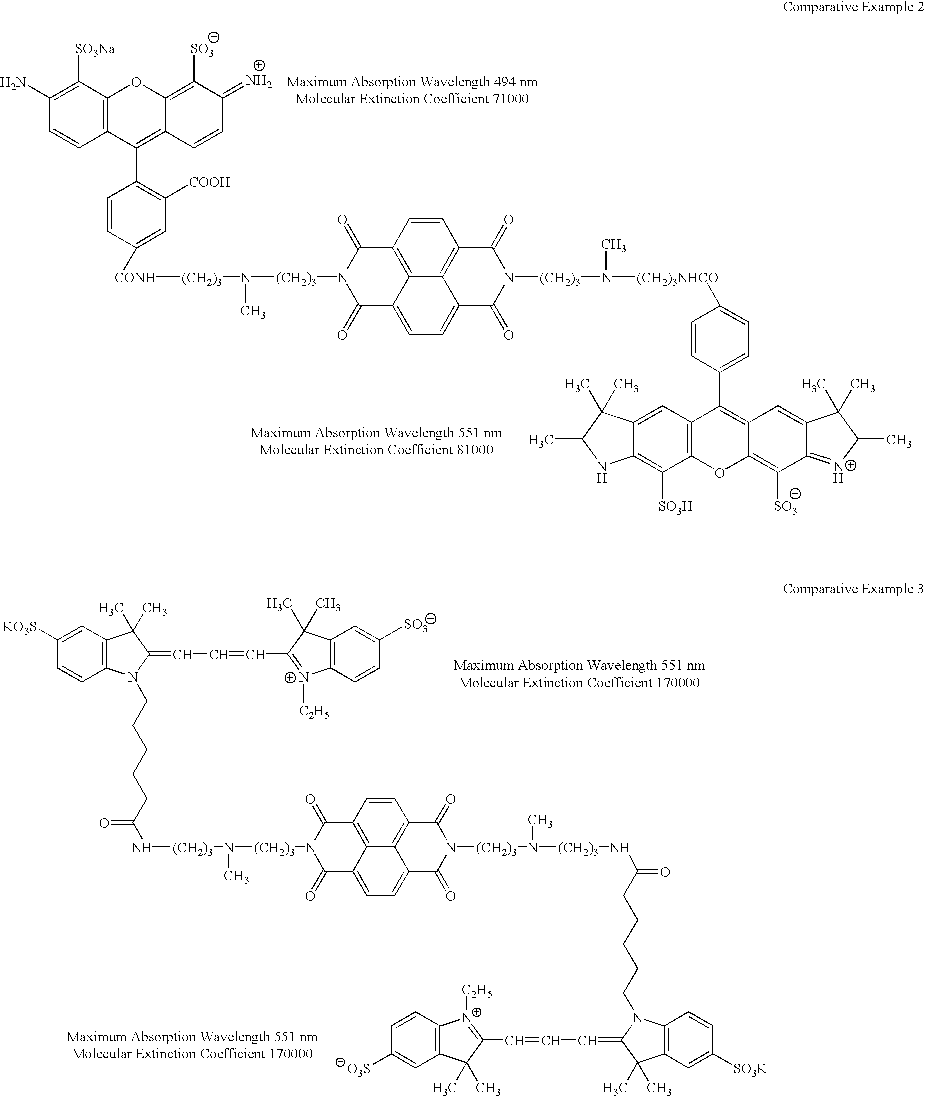

### Example 4

### Detection of DNA/RNA Hybrid

The same experiment as in Example 3 was performed except that 40-mer oligo-A was immobilized on the slide glass surface instead of the 60-mer DNA and oligo-U was used as the complementary sequence. As a result, results similar to those in Example 3 were obtained.

### Example 5

### Detection of DNA Hybrid

(1) Preparation of DNA Fragment-Immobilized Slide

Silane compound-coated slide (A) and VS-1-coated slide (B) were prepared in the same manner as in Example 1.

(2) Detection of Hybrid Multi-Stranded Nucleic Acid

An aqueous liquid containing DNA of SEQ ID NO: 1 mentioned in Sequence Listing, of which 3′ end was modified with an amino group, dispersed in 0.1 M carbonate buffer (pH 9.8, 1×10⁻⁶ M, 1 μL) was spotted on Slide (B) obtained in the above (1) to prepare Slide (C) Immediately after the spotting, the slide was left at 60° C. and humidity of 90% for 1 hour and then heated at 120° C. for 20 minutes. This slide was successively washed twice with a mixed solution of 0.1 mass % SDS (sodium dodecylsulfate) and 2×SSC (2×SSC: a solution obtained by diluting a stock solution of SSC 2-fold, SSC: standard saline citrate buffer) and once with a 0.2×SSC aqueous solution. Subsequently, the slide after the aforementioned washing was immersed in a 0.1 M glycine aqueous solution (pH 10) for 1 hour and 30 minutes, washed with distilled water and dried at room temperature to obtain Slide (D) on which the DNA fragments were immobilized.

60-mer DNA having a sequence complementary to the aforementioned DNA sequence was dispersed in a hybridization solution (mixed solution of 4×SSC and 10 mass % SDS, 20 μL), added with two kinds of compound with affinity to multi-stranded nucleic acids (1 μL each of 0.1 mM solution in dimethyl sulfoxide) and applied to Slide (D) obtained as above. After the surface of Slide (D) was protected with cover glass for microscope, the slide was incubated in a moisture chamber at 60° C. for 10 hours. Subsequently, this slide was washed with a mixed solution of 0.1 mass % SDS and 2×SSC, centrifuged at 600 rpm for 20 seconds and dried at room temperature. Fluorescent intensity of each slide glass was measured by a fluorescence scanning apparatus. Excitation was attained at a wavelength as close as possible to a maximum absorption wavelength of a dye of a shorter wavelength end, and the detection wavelength was controlled by adjusting a filter so as to obtain a maximum value. Further, the same experiment was performed without adding the 60-mer DNA having a complementary sequence.

In FIG. 2, fluorescence intensities of the samples are shown as values relative to fluorescence intensity of the sample of Comparative Example 4 with the addition of the complementary strand, which was taken as 100 (for each experiment, a compound of a shorter wavelength end was excited and luminescence of a compound of a longer wavelength end was detected). As a result, in Comparative Example 4, no signal was obtained without washing, because no fluorescence resonance energy transfer occurred. Whilst, signals were detectable in the samples obtained by the method of the present invention even without washing. Further, even after the washing, the backgrounds were low-and excellent S/N ratios were obtainable. In contrast, the background fluorescence intensity was found to be high in samples of the comparative example.

TABLE 2

| | | | Fluorescence |
|---|---|---|---|
| | | Fluorescence | intensity without |
| | | intensity of | complementary |
| Sample | Distinguishing ratio | signal | strand |
| Comparative | 1.2 | | |
| Example 4 | | | |
| (Compound a) | | | |
| Comparative | 3.1 | 100 | 47 |
| Example 4 | | | |
| (Compound b) | | | |
| Invention 5 | 3.5 | | |
| (Compound c) | | | |
| Invention 6 | 1.4 | | |
| (Compound a) | | | |
| Invention 6 | 5.2 | 198 | 36 |
| (Compound d) | | | |

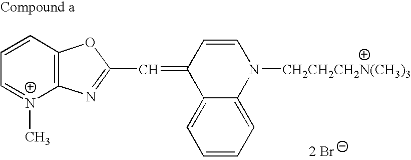

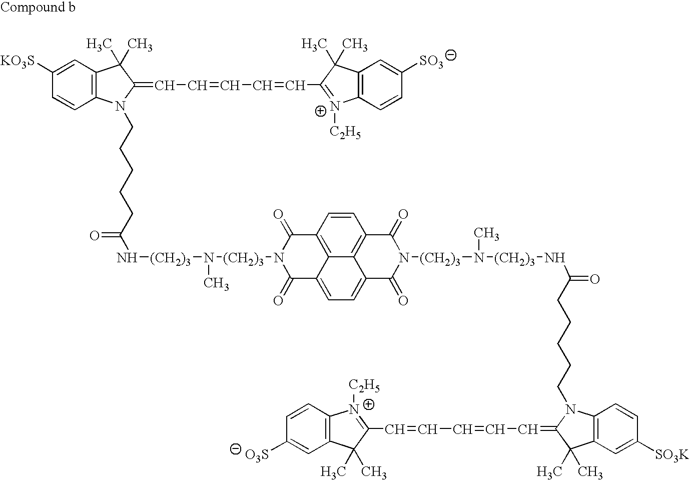

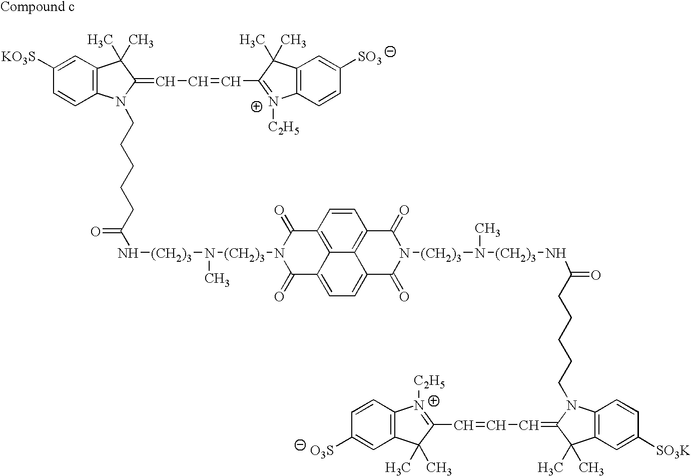

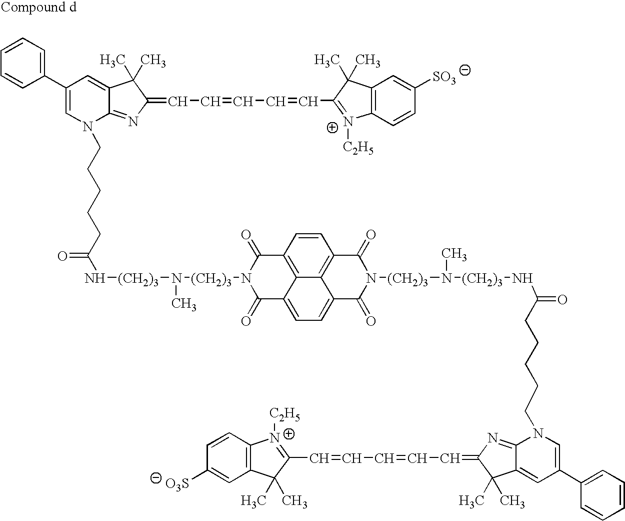

### Example 6

### Detection of DNA/RNA Hybrid

The same experiment as in Example 5 was performed except that 40-mer oligo-deoxy-A was immobilized on the slide glass surface instead of the 60-mer DNA and oligo-U was used as the complementary sequence. As a result, results similar to those obtained in Example 5 were obtained.

Sequence Listing

<110>Fuji Photo Film Co. Ltd.

<120>Method for detecting nucleic acid

<130>FA2118M/US

<160>1

<210>1

<211>60

<212>DNA

<213>Artificial

<400>1

GCTGCTGCTG GGCCAGTGGT TCCTCCATGT CCGGGGAGGA TCAGACACTT CAAGGTCTAG 60

What is claimed is:

1. A method for detecting a hybrid multi-stranded nucleic acid sample nucleic acid, which comprises the step of allowing the sample nucleic acid to interact with a probe nucleic acid to form a hybrid multi-stranded nucleic acid by hybridization of the probe nucleic acid and the sample nucleic acid, which further comprises the steps of allowing two or more kinds of compounds with affinity to multi-stranded nucleic acids, each having a different luminescent characteristic, to interact with the hybrid multi-stranded nucleic acid, and then detecting luminescence generated as a result of an energy transfer between the two or more kinds of the compounds with affinity to multi-stranded nucleic acids.

2. The method according to claim 1, wherein the probe nucleic acid is immobilized on a solid phase carrier.

3. The method according to claim 2, the luminescence is detected without washing after the hybrid multi-stranded nucleic acid and the compound with affinity to multi-stranded nucleic acids are allowed to interact to each other.

4. The method according to claim 1, wherein the energy transfer is a fluorescence resonance energy transfer.

5. The method according to claim 4, wherein a difference between wavelengths at maximum values in excitation spectrum and emission spectrum of the luminescence generated as a result of the energy transfer is expanded than a difference between wavelengths at maximum values in excitation spectrum and emission spectrum of each of said compounds with affinity to multi-stranded nucleic acid.

6. The method according to claim 4, wherein the luminescence is detected in which the difference between wavelengths at maximum values in excitation spectrum and emission spectrum is 80 am or more.

7. The method according to claim B, wherein the luminescence is detected in which the difference between wavelengths giving maximum values in excitation spectrum and emission spectrum is 100 nm or more.

8. The method according to claim 1, wherein two or more kinds of the compounds with affinity to multi-stranded nucleic acids are used wherein each of which has a chromophore with a molecular extinction coefficient of 70,000 or more.

9. The method according to claim 1, wherein at least one of the compounds with affinity to multi-stranded nucleic acids is represented by the following general formula (I):
(IC)-[(L)m-(SIG)q]n
wherein, IC represents a group having affinity for a multi-stranded nucleic acid; L represents a divalent linking group; SIG represents a chromophore which emits a detectable signal; “n” represents an integer of 2, 3 or 4; “m” represents 0 or 1; and “q” represents 0 or 1, provided that “q” is not 0 in all of the “n” pieces of (L)m-(SIG)q, and L, A, SIG and q may be the same or different in the “n” pieces of (L)m-(SIG)q.

10. A compound represented by the general formula (I) defined in claim 6, which is used for the method according to claim 1.

11. A method for detecting a hybrid multi-stranded nucleic acid, which comprises the steps of allowing a sample nucleic acid to interact with a probe nucleic acid to form a hybrid multi-stranded nucleic acid by hybridization of the probe nucleic acid and sample nucleic acid, and allowing a compound with affinity to multi-stranded nucleic acid to interact with the hybrid multi-stranded nucleic acid and detecting luminescence generated from said compound to detect the hybrid multi-stranded nucleic acid, wherein the compound with affinity to multi-stranded nucleic acid is a luminescent compound having two or more kinds of chromophores, each having a different luminescent characteristic, and at least two or more kinds of the chromophores among said chromophores have a difference of 80 nm or more between a maximum absorption wavelength of a chromophore of a shorter wavelength end and a maximum emission wavelength of a chromophore of a longer wavelength end.

12. The method according to claim 11, wherein the probe nucleic acid is immobilized on a solid phase carrier.

13. The method according to claim 11, wherein each of the at least two or more kinds of said chromophores has a molecular extinction coefficient of 70,000 or more.

14. The method according to claim 11, wherein the compound with affinity to multi-stranded nucleic acid is an intercalator.

15. A method for detecting a hybrid multi-stranded nucleic acid, which comprises the steps of allowing a sample nucleic acid to interact with a probe nucleic acid to form a hybrid multi-stranded nucleic acid by hybridization of the probe nucleic acid and the sample nucleic acid, and allowing two or more kinds of compounds with affinity to multi-stranded nucleic acids, each of said compounds has a distinguishing ratio of higher than 1 for a multi-stranded nucleic acid relative to a single-stranded nucleic acid and, to interact with the hybrid multi-stranded nucleic acid and detecting interaction between said compounds to detect the hybrid multi-stranded nucleic acid, wherein at least one kind of the compound among said compounds has the distinguishing ratio of 5 or more, or each of at least two kinds of the compounds among said compounds has the distinguishing ratio of 3 or more.

16. The method according to claim 15, wherein the probe nucleic acid is immobilized on a solid phase carrier.

17. The method according to claim 15, wherein at least one kind of the compounds among said compounds with affinity to multi-stranded nucleic acids is a luminescent compound.

18. The method according to claim 15, wherein at least one kind of the compound among said compounds with affinity to multi-stranded nucleic acids is an intercalator.
